(19) United States
(12) Patent Application Publication
(10) Pub. No.: US 2022/0132895 A1
(43) Pub. Date: May 5, 2022

(54) PROCEDURE FOR SELECTIVE EXTRACTION OF ACTIVE PRINCIPLED AND/OR OLEORESINS FROM VEGETABLE MATERIAL AND RELATED SYSTEM

(71) Applicant: Turatti S.R.L., Cavarzere (VE) (IT)

(72) Inventor: Antonio TURATTI, Cavarzere (VE) (IT)

(73) Assignee: Turatti S.R.L., Cavarzere (VE) (IT)

(21) Appl. No.: 17/434,772

(22) PCT Filed: Feb. 28, 2020
(86) PCT No.: PCT/IT2020/050044

§ 371 Date: Aug. 30, 2021

(30) Foreign Application Priority Data
Feb. 28, 2019 (IT) 102019000002929

(51) Int. Cl. A23L 27/10; B01D 11/02

(52) U.S. Cl. CPC A23L 27/11; B01D 11/0219; B01D 11/0296; B01D 11/0288; B01D 11/0253

(57) ABSTRACT

A process for the extraction of oleoresins from vegetable material by solvent comprising the following successive steps:

a solvent washing step of vegetable material in maceration means controlled in temperature and/or pressure for a time varying between 10 and 60 minutes, with a proportion between the mass of vegetable material to be treated and the volume of solvent between 1:5 kg/l 1:10 kg/l; the temperature is controlled by means of a cryogenic fluid and is between −60 ° C. and −100 ° C., thus obtaining a mixture containing a solid fraction, and a liquid fraction in which the oleoresins dissolved in the solvent are included;

a pressing step of the mixture in order to separate the solid fraction of vegetable material from the liquid fraction of solvent containing the oleoresins;

a step of filtration of the liquid fraction of the mixture;

a step of separation of the solvent from the liquid fraction of the mixture in an evaporator, controlled under pressure and temperature, in which the pressure is between 10 mbar and 50 mbar and the temperature is between 10° C. and 40° C., thus obtaining so the active principles and/or the oleoresins.

The present invention relates to a process for the extraction of active principles and/or oleoresins from vegetable material.

The invention also relates to a system for carrying out said process.

In the following, the description will be addressed to the treatment of specific vegetable materials, but it is to be understood that both the process and the system according to the invention can be used for the processing of different vegetable materials.

The invention is part of the field of systems for the selective extraction of active principles and aromatic components, such as essential oils, from vegetables, and in particular it refers to a process of extraction with solvent and to the obtainment of oleoresins/matrices resinous from vegetable material, and to a system suitable for the implementation of this process.

The process, and therefore the system, is capable of processsing different raw materials; by way of example but not limited, the following are cited:
Herbs/spices: basil, thyme, oregano, mint, lemongrass, cloves, chilli pepper, rosemary, anise.
Plants: eucalyptus, aloe vera, oak, pine, hops, moringa, cannabaceae, echinacea, helichrysum, lobelia, lavender, rose.
Fruit: coffee, vanilla, avocado, hazelnuts, maracuja, lemon, cedar, bergamot.

Currently the processes for separating oils, essences, perfumes or any other chemical compound of interest contained in a starting material are numerous and extremely varied, both as regards the parameters of the processes and the procedures and apparatus used.

The most common and used among them include:
Mechanical separation, in which the plant material is placed in a chamber and separated from the aromatic component by pressing.
Distillation, operated by evaporating a solvent (almost always water) with the raw material by immersion, to solubilize the oils within; the mixture is then cooled causing the separation of the hydrophobic oils from the water.
Extraction by solvent, in which the raw material is macerated in a suitable solvent for a variable time, allowing the solubilization of the oils, which are then separated by evaporation of the solvent; the types of solvents used may vary according to the compounds to be extracted, as well as the process parameters (pressure, temperature, time) and greatly influence the quantity and quality of the final product.
Enfleurage, the most ancient technique, where a grease is used instead of the solvent to absorb the odorous components of the raw material.
Filtering, a low efficiency technique if not combined with others, which uses mechanical means and/or membranes to obtain the separation of the solid part from the liquid part.

The processes of selective extraction of active principles from vegetable raw material are of great importance in many industrial sectors, for example in the parapharmaceutical and nutraceutical sector, and it has now become a primary necessity that of identifying and acting on the parameters of the processes that allow optimizing the production in terms of yield, quality and costs.

It is known that all industrially used extraction methods, both those using solvent and purely mechanical ones, tend to distort or alter in some way the perception and the properties of the obtainable final product, for a number of reasons. When choosing the method of extraction of a substance, and its process parameters, the goal remains to obtain a product of the best possible quality in an economically, and industrially convenient way. The use of solvents, the heat, the holding time of the components in solution before the separation are all factors that greatly affect the integrity of the molecules of the product, and therefore on its final quality.

In general, by increasing the temperature during extraction, or the maceration time of the raw material in the solvent, it is possible to separate a greater quantity of product, but there is also the risk of irreversibly deteriorating the delicate organic molecules that characterize it; doing so the impurities and/or unwanted substances extracted also increase.

Conversely, by decreasing the time and temperature of exposure to the solvent, the quantity of extracted oils becomes small, consequently increasing the costs and wastes of raw materials.

It is also possible using different solvents to facilitate the extraction of one or more substances according to the different chemical affinity, all obviously at the expense of the simplicity of realization of the apparatus and the identification of the most suitable process parameters.

By way of example, patent no. US 2017/15721344 describes a process of solvent extraction of cannabinoids from cannabis plants, using ethanol under subcritical conditions in a chamber maintained at pressure even higher than the atmospheric. Briefly, a process is described, which involves the processing of already dried cannabis plants, then subjected to washing with ethanol (with proportions of 1 L of ethanol per 1 kg of raw material) at higher than atmospheric pressure and temperatures between −40° C. and −50° C. The process includes numerous filtration steps, also with the aid of activated carbon and silica gel, which also provide for temperature variations.

The evaporation step of US'344, on the other hand, involves the achievement of temperatures in rotovapor up to 43-49° C., and is followed by further specific purifications for substances extracted from cannabis.

In light of the above, the invention in question differs from the known art cited in that it provides a process for treating vegetable material capable of extracting the oleoresins thereof in a simple and direct way, avoiding to carry out multiple filtering steps and with the use of tools such as silica gel and activated carbon.

Furthermore, the temperatures reached by the solvent are lower, and allow to obtain better extraction results from a qualitative point of view.

In addition, the system for carrying out the process is simple to construct and capable of operating on the raw material in an easy to implement cycle.

Therefore, the invention described in this document has the objective of optimizing the extraction process of essential oils and/or oleoresins from vegetable materials, through the research and implementation of innovative process parameters; specifically the apparatus described below is based on a process that uses low pressure, low temperatures and low times of stay of the raw material in the solvent maintained under controlled conditions. In this way, it is therefore possible to obtain a greater quantity of product, of high purity and quality, which is at the same time advantageous to produce and economically sustainable. However, the purely extractive step takes place in very low temperature conditions and at ambient pressure or lower: the lower the temperatures, the less unwanted chlorophylls and waxes are extracted from the raw material, to the full advantage of the quality of the extracted resin, which, precisely because of its extractive quality, does not require complicated filtration steps.

In general, the apparatus requires a minimum number of components that are sufficient for the implementation of the process, i.e. at least one refrigerated storage tank for the solvent, a maceration tank (for example an autoclave), an evaporator, a vacuum generation system, a refrigeration system (via refrigeration group or liquid nitrogen) and a condensed solvent recovery system.

It is therefore object of the invention a process for the selective extraction of active principles and/or oleoresins from vegetable material by means of solvent; this process comprises the following successive steps:
a. a washing step by solvent of the vegetable material in maceration means controlled in temperature and/or pressure for a time varying between 10 and 60 minutes, with a proportion between the mass of vegetable material to be treated and the volume of solvent between 1:5 kg/l and 1:10 kg/l,
wherein the temperature is controlled by means of a cryogenic fluid and is between −60° C. and −100° C.,
thus obtaining a mixture containing a solid fraction and a liquid fraction,
the liquid fraction comprising said solvent in which the oleoresins are dissolved;
b. a pressing step of said mixture in order to separate the solid fraction of vegetable material from the liquid one of solvent containing the oleoresins;
c. a filtering step of the liquid fraction of said mixture;
d. a separation step of said solvent from the liquid fraction of said mixture in an evaporator, controlled by pressure and temperature,
wherein the pressure is between 10 mbar and 50 mbar and the temperature is between 10° C. and 40° C., obtaining the active principles and/or the oleoresins.

In a preferred embodiment of the process, the temperature of the washing step a) is between −65° C. and −80° C.

Preferably, the filtering step c) takes place through a filter having a weft of 25 μm (micrometers) or less.

The process can include, between step b) and step c), a step b1) of storage of said liquid fraction in intermediate storage means.

There may also be a step b2) of transferring the liquid fraction resulting from step b), towards the evaporator, having a duration of less than 5 minutes.

After said step d), a step d1) of condensation of said solvent can also be provided.

In a preferred embodiment of the invention, the condensed solvent is recovered and transferred to collecting means.

In addition, it is possible that the solvent condensed in step d1) is sent, in a step d2), at the beginning of step a) for its reuse.

In preferred embodiments of the process, before returning to step a), the condensed solvent can be stored in temperature and/or pressure controlled cryogenic storage means, where the temperature is between −85° C. and −100° C. .

In the process, after step d), a step of removing the oleoresins from the evaporator can be provided by the collecting means of the oleoresins themselves.

Furthermore, the used solvent can consist of one or more alcohols.

The treated vegetable material can be very varied, and can comprise one or more of the following vegetable species: basil, thyme, oregano, mint, lemongrass, cloves, chilli pepper, rosemary, anise, eucalyptus, aloe vera, oak, pine, hops, moringa, cannabaceae, echinacea, helichrysum, lobelia, lavender, rose, coffee, vanilla, avocado, hazelnuts, maracuja, lemon, cedar, bergamot.

A further object of the present invention is also a system for carrying out the process described above.

An example of such a system may include in particular one or more of the following apparatuses suitably connected to each other:
means for storing a cryogenic fluid, connected to
means for collecting the solvent, equipped with a cooling jacket and connected to some
means for macerating the plant material with the solvent, additionally connected to the cryogenic fluid tanks and cooled thereby,
pressing means, cooled and arranged between the maceration means and the evaporator, for separating the liquid fraction from the solid fraction of the mixture,
filtration means, cooled and arranged between the pressing means and the evaporator,
an evaporator, arranged downstream of the maceration and/or pressing means, for the separation of the solvent from the active principles and/or from the oleoresins,
means for taking or collecting the active ingredients and/or oleoresins from the evaporator.

The system can comprise, in preferred embodiments, intermediate storage means of the liquid fraction of the mixture, which are also cooled, positioned between the pressing means and the filtering means.

The filtration means can be made of inert materials and resistant to chemical agents, for example polytetrafluoroethylene (PTFE) or stainless steel.

Furthermore, solvent condensation means can be provided downstream of the evaporator.

The system can be controlled under pressure through at least one pressure parameter control device; in particular, this control will be applicable to the cryogenic solvent storage means and/or the cryogenic fluid storage means and/or the maceration tank and/or the pressing means and/or the intermediate storage means and/or the filtering means and/or the evaporator.

The condenser located downstream of the evaporator can be regulated in temperature by means of a suitable condensate fluid, cooled by a refrigeration group exclusively dedicated to it.

In the system mechanical mixers or agitators can be provided, positioned inside the maceration means and/or the cryogenic solvent tanks.

Finally, the described system and/or process can be implemented in a vegetable material treatment line of the most varied types.

The present invention will now be described for illustrative but not limitative purposes, according to some specific embodiments thereof, with particular reference to the figures of the attached drawings, in which:

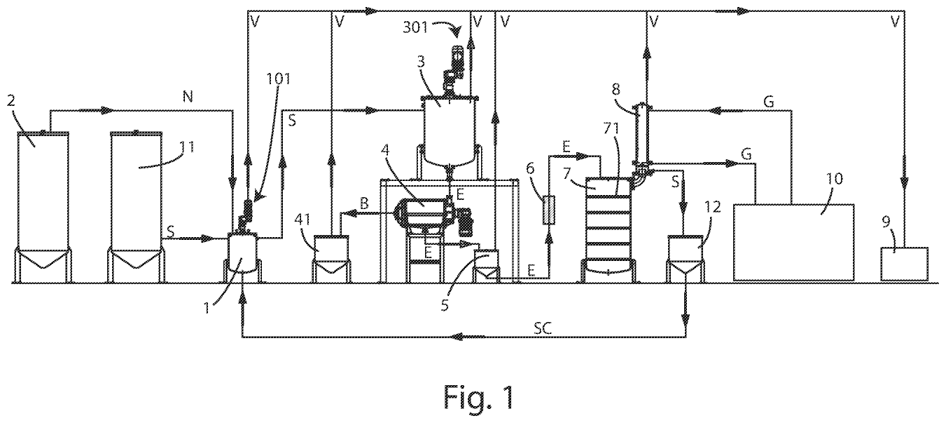

FIG. 1 is a schematic view of a system according to the invention.

With reference to FIG. 1, a preferred embodiment of the system according to the invention is shown, in particular for the processing of hops and/or cannabaceous plants.

In particular, in the system, generally indicated by the numerical reference 100, there are provided:
a cryogenic solvent tank 1, for containing a solvent and cooling it, in which it is possible to provide a stirrer 101;
one or more virgin solvent collecting tanks 11, connected to tank 1;
a storage tank 2, also connected to the tank 1, for the containment of a suitable cryogenic fluid, for example liquid nitrogen;
a maceration tank 3, with a liftable lid, inside which the vegetable material is inserted, for example by means of a conveyor belt (not shown).

This maceration tank 3 is connected directly to the cryogenic tank 1.

The vacuum group moves the fluids and/or the different substances from one container to the next during the following operations.

Following the supply of the solvent, a mixture is created inside the tank 3, placed in constant recirculation by means of a mechanical mixer 301, with the aim of ensuring suitable uniformity of the product resulting from the maceration and extraction operations. The tank 3 can integrate an ultrasound cavitator inside (not shown);
a press 4, for example of the mono-helical or bi-helical screw type, connected to the maceration tank 3 and used to separate by compression the liquid fraction (solvent in which the oleoresins are dissolved) of the mixture from the solid fraction (vegetable material);
a container 41 communicating with the press 4 and used to collect the solid fraction (residual or exhausted biomass) resulting after pressing;
tank 5, connected to the press 4, where to collect the liquid fraction coming out from the press itself;
a filter 6 downstream of the tank 5, when present, but in any case downstream of the maceration tank 3 and upstream of a;
evaporator 7, connected to;
a condenser 8, to condense the solvent at a temperature of about −12° C., connected downstream of the evaporator 7;
a tank for the condensed ethanol 12, placed upstream of the cryogenic tank 1 and downstream of the condenser 8, to which part of the solvent stored in the tank 12 is sent;
a vacuum generator or vacuum group 9, connected directly or indirectly to the tanks 1, 2, 3, 41, 5, 11 and 12, to the press 4, to the filter 6, to the evaporator 7 and to the condenser 8, which moves the fluids and/or the mixtures treated during the process, through the action of the depression generated in the destination tank of the fluids and/or in the tanks connected to the destination tank itself;
a refrigeration group or refrigeration group 10 serving the condenser 8.

A cycle of operation of the system 100 is now described in more detail and by way of example.

The solvent, for example ethanol (but for some vegetable materials it could be simply water) is stored in the collecting tank 11, at atmospheric pressure and at room temperature, or in any case at a suitable temperature (between the solidification T and the boiling T of the solvent), so as to prevent the evaporation.

Through the vacuum group 9, the solvent, which is not yet at the desired temperature, is transferred from the collecting tank 11 to the cryogenic tank 1.

Liquid nitrogen is circulated in a jacket or coil (not shown) integrated in the cryogenic tank 1 to cool the solvent; subsequently, the same nitrogen, which is now in the gas phase but still retains a certain cooling power, is introduced into the maceration tank 3 to cool the biomass and increase the efficiency of the system and the purity of the extract.

In the tank 1 the cooling step begins; in the embodiment shown in the figure, the temperature can vary in a range from −60° C. to −100° C.

Preferably, the temperature of the solvent is around a value between −90° C. and −95° C., indicated for the extraction of active principles from hops and/or cannabaceous plants.

The temperature range between −90° C. and −95° C. is preferably indicated for the extraction of raw materials in the maceration tank 3.

For design reasons or to give particular characteristics to the final product, this interval can be modified; in the specific case, the temperature of the extraction step inside the maceration tank 3 is about −70° C.

The vacuum conditions, not only for this section, but also for all the components of the system that require it, are guaranteed by the vacuum generator 9.

In the described embodiment of the system, the vacuum is created in the steps of transfer of the solvent from the tank 1 to the maceration container 3, from the tank 5 to the evaporator 7, and during the evaporation step, to favor the complete separation of the solvent from the oleoresin; the same components indirectly place under vacuum those connected to them in an intermediate position.

In addition to tank 1 it is possible to provide further storage tanks (not shown) to cool an additional volume of solvent and have it available more quickly for the next cycle.

In a further embodiment it is possible to eliminate the tank 5, without the storage step of the mixture prior to the filtration.

In any case it is also possible, if necessary, increasing the number of intermediate tanks provided, be they for the solvent, for the mixture or other.

The raw material, in particular the vegetable material or biomass or solid fraction, in the example shown the hops or the cannabaceae, is loaded by means of a conveyor belt (not shown) into the maceration tank 3, which is also kept at a low temperature, as closer to the temperature of the cryogenic solvent.

The conveyor belt can have an integrated weighing system, in order to facilitate the loading operation of the biomass to be treated towards the maceration tank 3.

To limit the contamination of the extract due to the water contained in the biomass, it is preferable to use dry vegetable material; it is also preferable since the volume occupied by dry biomass is lower than in wet biomass, therefore more raw materials can be processed per cycle.

The maceration tank 3 has a sufficient capacity to treat the raw material with the solvent in variable proportions, between 1:3 and 1:15 (kg/l), preferably between 1:5 and 1:10 kg of biomass per liter of solvent. The treatment takes place by immersion, with a duration ranging from 10 to 60 minutes.

After refining the parameters to be used during the procedure, it was possible to reach a compromise between the cost of the procedure and the system (insulation of the pipes and of the containers, refrigerating power of the instrumentation) and maintaining the temperature around a stationary value for the entire duration of the wash: this value is around −70° C.

In this step, if present, the ultrasonic cavitator, immersed in the mixture, can be activated. A mechanical mixer mixes the biomass together with the solvent inside the maceration tank 3, in order to improve the extraction efficiency. The oily compounds to be extracted, contained in hops or in the cannabaceous plants, are dissolved in the solvent due to their greater chemical affinity.

In the example considered, the transfer of the biomass-solvent mixture from the maceration tank 3 to the press 4 takes place, on the other hand, by gravity, for example through a discharge system with opening on the bottom, which can be integrated into the tank 3 itself.

Subsequently, the mixture containing the residues of vegetable material and the solvent with the extracted active principles are separated: the discharge system, present on the bottom of the maceration tank 3 is opened, and the mixture is conveyed by gravity into the press 4, placed under the tank 3.

In this step, a part of the solvent in which the extracted active principles are dissolved is immediately evacuated and sent to the intermediate tank 5 for the intermediate storage; as the press 4 gradually compresses the biomass, the fraction of remaining solvent is also obtained and conveyed to the tank 5.

The exhausted biomass, deprived of the liquid fraction, is then expelled from the press 4 and sent to the container 41 for subsequent disposal or for any subsequent less noble operations.

Subsequently, the solvent containing the oleoresin is directed towards the evaporator 7 through a filter 6; such filter 6 may be, for example, in stainless steel, PTFE or other equivalent material, preferably with a weft of 25 μm or less.

The passage of the mixture into the filter 6 is also carried out with the aim of retaining any foreign bodies deriving from the washing of the raw material.

The process according to the invention, carried out with the system 100 illustrated in FIG. 1, provides for the separation from the liquid component of the mixture of the oleoresin component and/or of the active ingredients, which will constitute the final product, and the solvent, which will be instead reused for subsequent processing cycles.

Furthermore, the peculiar characteristic of the process object of the present invention, and which distinguishes it from other procedures based on the same extraction principle, is the transfer speed with which the mixture is conveyed from the maceration tank 3 to the tank 5, with a time of the order of a few minutes, preferably less than 10 minutes; even more preferably, the duration of the transfer step will be less than 5 minutes.

More in detail, the transfer step previously described refers to the passage of the liquid fraction into the filter 6; it is important to quickly bring the liquid fraction to the evaporator 7.

By minimizing the duration of the step of transfer of the liquid fraction to the evaporator, its temperature can be kept low.

In this way, the degradation of dissolved substances is avoided and the purity of the final product is preserved.

In this perspective, the maintenance of the vacuum in the various components of the system limits the possibility of thermal exchanges with the external environment, to the full advantage of the quality of the final product.

If, in an alternative embodiment of the system, this tank 5 is not present, the step of passage through the filter 6 may have the minimum duration already mentioned.

The transfer rate must be kept low in order to ensure that the first portion of removed solution from the maceration tank 3 is almost equivalent in terms of extractive contents to the last extracted portion, without therefore compromising the homogeneity of the solvent-oleoresin liquid fraction.

By means of an appropriate system design, it is possible to obtain that the transfer takes place in the times described; some possible measures to be put in place to achieve this feature are, for example:
varying the dimensions of the solvent tanks 1, 11, 12, of the macerator 3 for the biomass-solvent mixture, of the evaporator 7, and the like, so as to control the maximum quantities of products used;
consequently correct the dimensions of the valves and of the connection pipes between the different containers, so as to be capable of working with different flow rates according to the case;
if possible, act on the proportions and/or on the quantities of solvent and vegetable material used in the process.

The transfer speed is a peculiar feature of the process object of the invention, essential for obtaining a product with high quality and final characteristics, as well as for improving the final yield of the entire process (i.e. to increase the quantity of product obtained starting from the same raw material treated).

In other words, the process and the system according to the invention make it possible to increase the quantity of final product obtained, without however compromising its quality but, on the contrary, improving it.

The effective refining of the raw mixture thus obtained is then completed after the transfer into the evaporator 7, where the combination of low pressure conditions (preferably between 10 mbar and 50 mbar) and relatively low temperature (preferably lower than 40° C., even more preferably around 20° C.) allows the separation of the solvent from the oleoresin and/or from the active principles, which therefore collect on the bottom of the evaporator 7.

In the described embodiment, the evaporator 7 integrates one or more removable trays 71, made for example of stainless steel and arranged on several levels, where at the end of the process the oleoresin, i.e. the non-evaporable component, is collected.

The oleoresin collected in the trays 71 can at this point be recovered with suitable extraction means, manual or automatic.

The evaporated solvent is condensed through the exchanger 8, which is connected to the evaporator 7 and which works through a suitable condensate fluid, preferably water mixed with a glycol, or glycol water; this fluid is at a temperature of about −12° C.

The solvent is then collected, in a preferred embodiment according to the invention, in a tank for the condensed solvent 12.

This tank 12 is connected to the cryogenic tank 1, since the condensed solvent can be subsequently transferred to the cryogenic tank 1 to be cooled again and used for further cycles, possibly together with a part of virgin solvent.

It is advisable storing the virgin solvent separately from the condensed one after the extraction, since the condensed solvent is more susceptible to degradation as it is gradually used in an increasing number of cycles.

The connections among the different components of the system are shown in FIG. 1; in particular, those necessary to guarantee the execution of the procedure are illustrated below.

The direct connections between the different components of the system are illustrated below:
the letter N indicates the connections with the cryogenic fluid containers, in this case liquid nitrogen, to maintain a sufficiently low working temperature;
the letter S indicates the transit pipes of the solvent only, whether cooled or not, before entering the maceration tank 3;
the letter V identifies the connections for the maintenance of the vacuum and the transfer of the different substances between the various containers, operated through the vacuum group 9;
the letter B indicates the pipe, through which the exhausted biomass passes, leaving the press 4 and directed towards the tank or container 41 of the residual biomass;
the letter E indicates the passage pipes of the extract, that is of the liquid fraction containing the oleoresins separated from the solid fraction (exhausted biomass) by means of the press 4;
the letter G indicates the connection between the refrigeration group 10 and the condenser 8, for supplying the condensate fluid;
finally, SC indicates the transfer line from the condensed solvent tank 12, collected after the separation of the oleoresins in the condenser 8, towards the cryogenic tank 1.

It is to be understood that in the connection lines between the various components, and especially in the vacuum lines V, valves or other flow control means are interposed where appropriate, which allow to include or exclude some components of the system from the application of certain pressure and/or temperature conditions.

The transfer of the liquid solvent from the tank 12 to the tank 1 can be carried out by means of a suitable pump (not shown in the figure).

The present invention has been described, for illustrative but not limitative purposes, according to its preferred embodiments, but it is to be understood that variations and/or modifications may be made by those skilled in the art, without thereby departing from the relative scope of protection.

In the claims:

1. Process for the selective extraction of active principles and/or oleoresins from vegetable material by solvent, characterized in that it comprises the following successive steps:
a. a washing step by solvent of the vegetable material in maceration means (3) controlled in temperature and/or pressure for a time varying between 10 and 60 minutes, with a proportion between the mass of vegetable material to be treated and the volume of solvent between 1:5 kg/l and 1:10 kg/l, wherein the temperature is controlled by means of a cryogenic fluid and is between −60° C. and −100° C.,
thus obtaining a mixture containing a solid fraction and a liquid fraction,
the liquid fraction comprising said solvent in which the oleoresins are dissolved;
b. a pressing step of said mixture in order to separate the solid fraction of vegetable material from the liquid one of solvent containing the oleoresins;
c. a filtering step of the liquid fraction of said mixture;
d. a separation step of said solvent from the liquid fraction of said mixture in an evaporator (7), controlled by pressure and temperature,
wherein the pressure is between 10 mbar and 50 mbar and the temperature is between 10° C. and 40° C., obtaining the active principles and/or the oleoresins.

2. Process, according to claim 1, characterized in that the temperature of the washing step a) is between −65° C. and −80° C.

3. Process, according to claim 1, characterized in that said filtering step c) takes place by means of a filter having a weft of 25 μm (micrometers) or less.

4. Process, according to claim 1, characterized in that between the step b) and the step c) there is a step b1) for storaging said liquid fraction in intermediate storage means (5)

5. Process, according to claim 1, characterized in that it provides a step b2) of transferring said liquid fraction resulting from the step b) to said evaporator (7), having a duration of less than 5 minutes.

6. Process, according to claim 1, characterized in that it comprises, after said step d), a step dl) of condensation of said solvent.

7. Process, according to claim 6, characterized in that said condensed solvent is recovered and transferred into storage means for the condensed solvent (12).

8. Process, according to claim 6, characterized in that said solvent condensed in said step d1) is sent, in a step d2), at the beginning of the step a) for its reuse.

9. Process, according to claim 8, characterized in that said condensed solvent is sent in cryogenic storage means (1) before resending it to said step a), said cryogenic storage means (1) being controlled in temperature and/or pressure, where the temperature is between −85° C. and −100° C.

10. Process, according to claim 1, characterized in that it comprises, after said step d), a step of removing the oleoresins from said evaporator (7) by means of collecting means (71) of said oleoresins.

11. Process, according to claim 1, characterized in that said solvent consists of one or more alcohols.

12. Process, according to claim 1, characterized in that said vegetable material can comprise at least one of the following:
basil, thyme, oregano, mint, lemongrass, cloves, chilli pepper, rosemary, anise, eucalyptus, aloe vera, oak, pine, hops, moringa, cannabaceae, echinacea, helichrysum, lobelia, lavender, rose, coffee, vanilla, avocado, hazelnuts, maracuja, lemon, cedar, bergamot.

13. System for carrying out the process according to claim 1.
